(19) United States
(12) Patent Application Publication
(10) Pub. No.: US 2015/0068521 A1
(43) Pub. Date: Mar. 12, 2015

(54) CONTAINER AND PHARMACEUTICAL PRODUCT

(71) Applicants: BAXTER HEALTHCARE SA, Glattpark (CH); BAXTER INTERNATIONAL INC., Deerfield, IL (US)

(72) Inventors: David D. Hsu, Evanston, IL (US); Simon Freed, Providence, RI (US)

(21) Appl. No.: 14/022,201

(22) Filed: Sep. 9, 2013

(51) Int. Cl. A61M 16/18; A61K 31/02; A61M 16/08; A61M 16/20

(52) U.S. Cl. CPC A61M 16/186; A61M 16/20; A61K 31/02; A61M 16/0816
USPC 128/203.12

(57) ABSTRACT

An anesthetic container includes an anesthetic receptacle and a valve assembly attachable to the receptacle. The valve assembly includes a valve seat with a first sloped surface and a valve member with a second sloped surface, the first and second sloped surfaces having different slopes. The first and second sloped surfaces abut each other to form a gasketless, fluid-tight seal therebetween with the valve assembly in a closed configuration. The valve member is spaced from the valve seat in an open configuration to permit the flow of the anesthetic from the receptacle. The valve assembly may also include an exterior groove and a toroidal sealing member disposed in the groove, the toroidal sealing member having an X-shaped cross-section.

### BACKGROUND

This patent is directed to an anesthetic container and a pharmaceutical product, and, in particular, to an anesthetic container and a pharmaceutical product to be used with a halogenated anesthetic.

Liquid anesthetic is conventionally shipped from the manufacturer to the user (medical professional, hospital, etc.) in a container. While the design of the container may vary, it is often the case that the container will include an adapter with a valve assembly disposed in the neck of the container. The valve assembly controls the flow of liquid anesthetic from the container into a vaporizer, where the liquid anesthetic vaporizes typically in the presence of a carrier gas. The valve assembly may also control the return flow of vapor from the vaporizer into the container as the liquid is displaced from the container.

U.S. Patent Application No. 20060048842 illustrates one example of such a valve assembly (or valve, for short). A valve member and an associated cylindrical partition move within a conduit to control the exchange of liquid anesthetic and anesthetic vapor with a vaporizer. The illustrated valve or valve assembly is held in place through the cooperation of a rim of a cage, a rim disposed about the container neck, and a crimped ferrule. To seal the connection between the rim of the cage and the rim of the neck, a deformable gasket is disposed between the opposing rims. While not illustrated, a deformable gasket typically is disposed on the valve member, to seal the valve member to the conduit when the valve member is in the closed position. In addition, an o-ring is disposed about the valve assembly to seal the exterior interface between the valve assembly and the vaporizer port.

While different configurations of valve assembly have been tried over the years, it has remained conventional practice to place a gasket between the valve assembly and the container, and between the valve member and the conduit. Loss of seal between the valve assembly and the neck of the container, or between the valve member and the conduit, could have a multiple negative consequences. For example, leakage of anesthetic from the container into the environment is generally undesirable. Additionally, leakage of anesthetic represents a loss of value to the customer and to the supplier, from whom the customer is likely to seek compensation for the lost product.

However, the presence of the sealing gaskets in the assembly is not without its own set of consequences. The additional part count represented by the inclusion of these gaskets may add to the manufacturing costs and complexity. Further, each part within the assembly must be verified for safety, relative to its use in a medical device or system. Additionally, each part within the assembly must be verified for quality, relative to its intended use.

Moreover, the o-ring used to form the seal between the valve and vaporizer port must also be verified for safety and quality. The o-ring is subject to significant forces to deform the o-ring, and thereby provide a secure, fluid-tight seal. These forces are also applied to the o-ring when the valve is advanced into and withdrawn from the vaporizer port, which wears the o-ring and reduces the life of the o-ring.

As set forth in more detail below, the present disclosure sets forth a container with an improved valve assembly embodying advantageous alternatives to the valve assemblies of prior art devices.

### SUMMARY

In one aspect, an anesthetic container includes a receptacle for holding an anesthetic and having a wall defining an interior space and a passage in fluid communication with the interior space, and a valve assembly that is attachable to the receptacle to control flow of anesthetic through the passage to a vaporizer. The valve assembly includes a valve seat with a first sloped surface and a valve member with a second sloped surface, the first sloped surface and the second sloped surface having different slopes. The first and second sloped surfaces abut each other to form a gasketless, fluid-tight seal therebetween with the valve assembly in a closed configuration to limit the flow of the anesthetic from the receptacle. The valve member is spaced from the valve seat with the valve assembly in an open configuration to permit the flow of the anesthetic from the receptacle.

In another aspect, an anesthetic container includes a receptacle for holding an anesthetic and having a wall defining an interior space and a passage in fluid communication with the interior space, the anesthetic selected from the group of halogenated anesthetics consisting of sevoflurane, desflurane, isoflurane, enflurane, methoxyflurane and halothane. The anesthetic container also includes a valve assembly that is attachable to the receptacle to control flow of anesthetic through the passage to a vaporizer. The valve assembly includes a conduit having a wall defining a conduit passage with a first end and a second end, a valve seat defined at the second end of the conduit passage and a valve member, the valve member and the valve seat abutting each other in a closed configuration to limit the flow of the anesthetic from the receptacle, and spaced from each other with the valve assembly in an open configuration to permit the flow of the anesthetic from the receptacle. The wall of the conduit has an external surface with a groove formed therein at the first end, and further comprising a toroidal sealing member disposed in the groove, the toroidal sealing member having an X-shaped cross-section.

Additional aspects of the disclosure are defined by the claims of this patent.

### BRIEF DESCRIPTION OF THE DRAWINGS

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings is necessarily to scale.

FIG. 1 is a fragmentary, cross-sectional view of a container according to the present disclosure, with a valve assembly in a closed configuration;

FIG. 2 is an enlarged, partial cross-sectional view of a container according to the present disclosure, highlighting the interface between the valve member and the valve seat;

FIG. 3 is a partial cross-sectional view of the valve assembly of FIG. 1, with the receptacle and the ferrule removed and the valve assembly in an open configuration;

FIG. 4 is a perspective view of the conduit of FIG. 1; and

FIG. 5 is a partial cross-sectional view of the toroidal sealing member used with the conduit of FIG. 1.

### DETAILED DESCRIPTION OF VARIOUS EMBODIMENTS

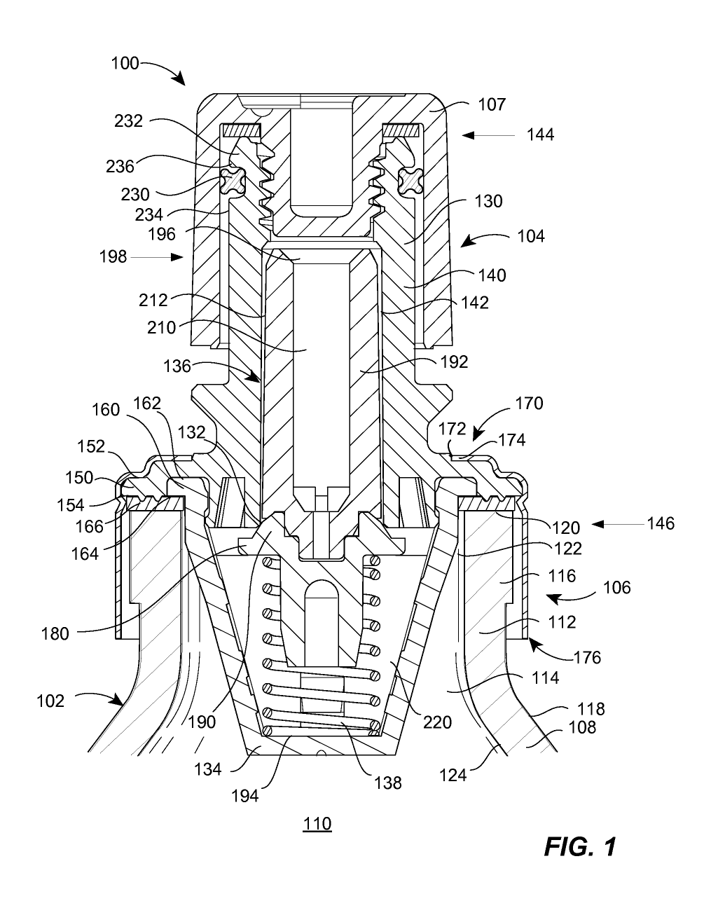

FIG. 1 illustrates an embodiment of an anesthetic container 100. The container 100 may be used with halogenated inhalation anesthetics such as sevoflurane (fluoromethyl 2,2,2-trifluoro-1-[trifluoromethyl]ethyl ether), desflurane (1,2,2,2-tetrafluoroethyl difluoromethyl ether), isoflurane (1-chloro-2,2,2-trifluoroethyl difluoromethyl ether), enflurane (2-chloro-1,1,2-trifluoroethyl-difluoromethyl ether), methoxyflurane (2,2-dichloro-1,1-difluoroethyl methyl ether) and halothane (2-bromo-2-chloro-1,1,1-trifluoroethane), for example, which may be disposed in the container to define a pharmaceutical product. According to certain embodiments, the anesthetic may be selected from a group consisting of sevoflurane, desflurane, isoflurane, enflurane, methoxyflurane and halothane. All of these halogenated anesthetics may be liquids under ambient conditions. The container 100 may be used to store these anesthetics for prolonged periods of time (e.g., weeks, months, or years), and thus may be referred to as a storage container.

The container 100 includes a receptacle 102 for holding the anesthetic, a valve assembly 104, and a ferrule (or cover) 106. The container 100 may also include a cap 107 that is fitted over the end of the valve assembly 104, as illustrated. The cap 107 may provide a barrier to anesthetic loss as well as limiting access to and contamination of the valve assembly 104. A sealing gasket (which may be made of low density polyethylene (LDPE)) may be disposed between the valve assembly 104 and the cap 107.

The receptacle 102 (which may in the form of a bottle as illustrated) has a wall 108 that defines an interior space 110 and a neck 112 with a passage 114 in fluid communication with the interior space 110. The anesthetic may be disposed in the interior space 110, so as to be contained within the receptacle 102. As illustrated in FIG. 1, the embodiment of the receptacle 102 has a neck 112 with a smaller cross-section than the widest part of the receptacle 102; this need not be the case according to all embodiments of the present disclosure. In addition, the receptacle 102 has a flange 116, preferably positioned at the neck 112 of the receptacle 102. As illustrated in FIG. 1, the flange 116 may depend from an exterior surface 118 of the wall 108 to define a rim 120 about an opening 122 in communication with the passage 114 through the neck 112.

According to an embodiment of the present disclosure, the receptacle 102 is made of glass. According to other embodiments, the receptacle 102 may be made of metal, for example, steel, aluminum or an aluminum alloy; according to still other embodiments, the receptacle 102 may be made of a polymer, such as polyethylene terephthalate (PET). Furthermore, according certain embodiments, a polymer or other material may be applied as a thin layer to define the exterior surface 118 of the wall 108. Additionally, according to further embodiments, a polymer or other material may be applied as a thin layer to define an interior surface 124 of the wall 108.

The valve assembly 104 is attachable or is attached to the receptacle 102 to control flow of fluids (e.g., anesthetic) through the passage 114 out of (and in to) the receptacle 102 to (and from) a vaporizer. The valve assembly 104, as illustrated, includes a conduit 130, a valve seat 132, a cage (or basket) 134, a valve member (or core) 136, and a resilient or biasing member 138. Other elements may be included as well, but as explained in greater detail below, the valve assembly 104 is designed to be gasketless in regard to the interface between the valve member 136 and the conduit 130, or more particularly between the valve member 136 and the valve seat 132.

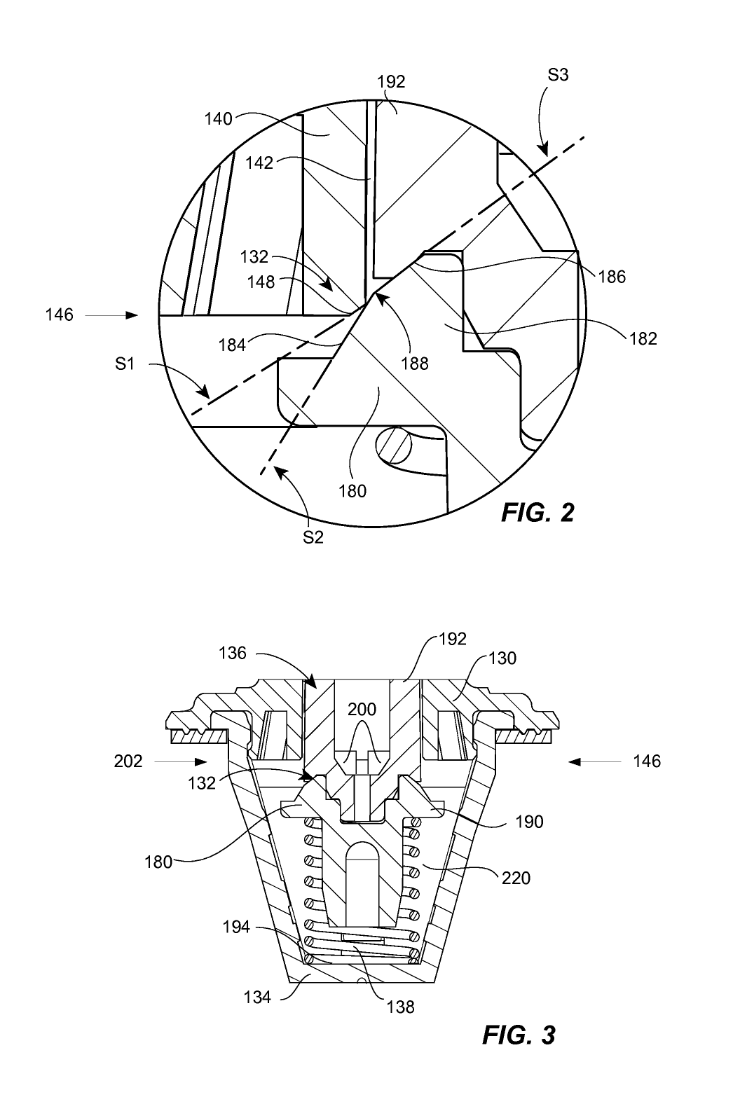

Beginning then with the conduit 130, the conduit 130 has a cylindrical wall 140 that defines a passage 142 with a first end 144 and a second end 146. The cylindrical wall 140 may have a frusto-conical surface 148 (see FIG. 2) disposed about the second end 146 of the passage 142. The surface 148 may define the valve seat 132, the further details of which will be described in greater detail below; the valve seat 132 may thus be described as defined at the second end 146 of the conduit passage 142 according to the illustrated embodiment. The conduit 130 also includes a conduit flange 150 (see FIG. 1) that depends outwardly from the wall 140. The conduit flange 150 is disposed proximate to the second end 146 of the passage 142, as illustrated. The conduit flange 150 has opposing first and second surfaces 152, 154.

The cage 134 (which may be made of nylon (e.g., nylon 66)) has a cage flange 160 that is disposed between the conduit flange 150 and the neck 112 of the receptacle 102. In particular, the cage flange 160 has opposing first and second surfaces 162, 164, and the first surface 162 of the cage flange 160 abuts the second surface 154 of the conduit flange 150, while the second surface 164 of cage flange 160 abuts a gasket 166 (which may be made of low density polyethylene (LDPE)) disposed on the rim 120 of the receptacle 102 (i.e., between the conduit flange 150 and/or cage flange 160 and the neck 112 of the receptacle 102, or more particularly, the rim 120 of the receptacle 102). A fluid-tight seal may be formed as a consequence.

Even though a fluid-tight seal may be formed, a layer of resilient polymer may define the exterior surface 118 of the receptacle 102, at least in the region of the neck 112 of the receptacle 102. For example, a layer of low-density polyethylene may be disposed on a layer of aluminum or aluminum alloy in the region of the neck 112 of the receptacle 102. According to certain embodiments, the low-density polyethylene may be applied using powder coating techniques, in particular where the wall 108 of the receptacle 102 is includes a layer of aluminum or an aluminum alloy. Other methods of including or applying the low-density polyethylene may also be used.

It should be noted that the entirety of the wall 108 of the receptacle 102 need not include the layer of low-density polyethylene. For example, it may be that other regions of the wall 108 of the receptacle 102 include other polymers, as discussed above. According to an exemplary embodiment, the receptacle 102 may include a wall 108 with a layer of a lacquer or an enamel disposed on a layer of aluminum or aluminum alloy, the layer of lacquer or enamel defining, at least in part, the interior surface 124 of the wall 108 of the receptacle 102. According to certain embodiments, the lacquer or enamel may include an epoxyphenolic resin.

As illustrated, the ferrule 106 is disposed over at least a portion of the conduit flange 150 and the cage flange 160 and about at least a portion of the neck 112 to attach the valve assembly 104 to the receptacle 102. The ferrule 106 has a cylindrical shape, with a first end 170 having an opening 172 with a rim 174 disposed about the opening 172 and defining the opening 172. The conduit 130 depends through the opening 172 in the first end 170 of the ferrule 106. The second end 176 of the ferrule 106 may be crimped about the flange 116 of the receptacle 102 to attach the valve assembly 104 to the receptacle 102. The ferrule 106 may be made of aluminum.

While the illustrated embodiment of the present disclosure has been illustrated with the valve assembly 104 attached to the receptacle 102 with a ferrule 106, it will be recognized that the container 100 need not be only defined as such. For example, the valve assembly 104 may be in the form of an adapter that is mated with the receptacle 102 only just prior to use, the passage 114 being closed instead through the use of a cap that may be threaded on to the receptacle 102 or held in place by a “snap-off” fit. According to such an embodiment, the cap is removed from the receptacle 102 prior to use, and the valve assembly 104 inserted into the passage 114 to attach the valve assembly 104 to the container. As such, the embodiment of the container 100 thus defined does not require the ferrule 106 illustrated in FIG. 1.

With reference to FIGS. 1 and 3, it will be recognized that the valve member 136 is disposed between the conduit 130 and the cage 134, or more particularly between the valve seat 132 and the cage 134. The valve member 136 has a first end 180 that abuts the valve seat 132 when the valve member 136 is in the closed position (as seen in FIG. 1). This may coincide with a closed configuration of the valve assembly 104. The valve member 136 is spaced from the second end 146 of the passage 142 and the valve seat 132 with the valve member 136 in the open position (FIG. 3). This may coincide with an open configuration of the valve assembly 104 that permits the flow of the anesthetic from the receptacle 102.

As illustrated in FIG. 2, the valve member 136 may have a head 182 formed at the first end 180. The head 182 may include at least one sloped surface 184, as illustrated. The sloped surface 184 faces the sloped surface 148 that defines the valve seat 132, and abuts the surface 148 to form a gasketless, fluid-tight seal therebetween with the valve assembly 104 in the closed configuration so as to limit the flow of the anesthetic from the receptacle 102. As illustrated, neither surface 148, 184 exhibits any curvature, such that the cross-section of the surfaces 148, 184 appears linear instead of exhibiting any arcuate section.

Elimination of a gasket between the surfaces 148, 184 is advantageous in that it eliminates the gasket material that comes into contact with the fluid, thereby eliminating a potential source of extractables or leachables that may impact the shelf life or other characteristic of the fluid. As observed by Schulte and Ellis, in Anesthesia and Analgesia, vol. 2, no. 2, pp. 644-645 (February 2010), a yellow discoloration may result from use of conventional anesthetic containers, which containers include the use of gaskets between valve members and valve seats. While they report that no patient harm is likely to result, the limitation or elimination of the compounds reported by Schulte and Ellis is believed to be possible through the elimination of gaskets in the anesthetic container.

As illustrated, the first sloped surface 148 of the valve seat 136 has a slope that is different than a slope of the second sloped surface 184 of the valve member 136. With reference to FIG. 2, a first line S1 has been extended from the sloped surface 148 and a second line S2 has been extended from the sloped surface 184 for ease of visualization. The first line S1 represents the slope of the surface 148, and the second line S2 represents the slope of the surface 184. With the valve seat 132 and valve member 136 oriented as illustrated in FIGS. 1-3, it will be recognized that the slope of line S2 is steeper than the slope of line S1; that is, the rise of the line S2 in the vertical direction is larger than the rise of the line S1 for a comparable run in the horizontal direction.

In addition, it will be recognized that the slopes of the first and second surfaces 148, 150 are not horizontal, nor are they vertical. Instead, the slopes are such that the angle formed between the surface and the horizontal is between 0° and 90°. For example, the slope of the surface 148 may be described as 45°, while the slope of the surface 184 may be described as 60°, with reference to FIG. 2.

It is believed that the valve seat 132 and the valve member 136, or more particularly the surfaces 148, 184, abut along a line of contact that runs about the circumference of the surface 184. It is also believed that the difference in the slopes of the surfaces 148, 184 permits a gap to be defined between sections of the facing surfaces 148, 184 disposed radially outward (relative to a longitudinal axis of the valve assembly 104) of the line of contact between the surfaces 148, 184. The gap between the sections of the facing surfaces 148, 184 may accommodate manufacturing tolerances in the surfaces 148, 184.

As also best illustrated in FIG. 2, the valve member 136 may have a third sloped surface 186. The third sloped surface may be disposed radially inward (again relative to the longitudinal axis of the valve assembly 104) of the second sloped surface 184, and the second and third sloped surfaces 184, 186 may meet at an interface 188 along adjoining edges. The interface 188 may be disposed radially inward (relative to the afore-mentioned longitudinal axis) of the line of contact between the first and second sloped surfaces 148, 184. The third sloped surface 186 may have a slope (represented by the line S3) that is not as steep as the slope of the second sloped surface 184.

With reference to FIGS. 1 and 3, it will be recognized that the valve member 136 includes a plate (or core seat) 190 and a tube (or core extension) 192, as illustrated. It will be recognized that other valve members may be designed wherein the plate 190 and tube 192 are formed as a single unit (i.e., integrally with each other). Thus, the illustrated embodiment is not intended to be limiting in this regard. The plate 190 may be made of low density polyethylene (LDPE), for example, while the tube 192 may be made of nylon (e.g., nylon 66).

The plate 190 defines the first end 180 of the valve member 136 with the surface 184, and occludes the second end 146 of the passage 142 through the conduit 130 with the valve member 136 in the closed position. The plate 190 may be referred to as a poppet valve. The valve member 136 is biased towards a closed position, illustrated in FIGS. 1 and 3, through the action of the resilient member 138, which may be a spring, as illustrated. The spring 138 may be made of stainless steel. The resilient member 138 is disposed between the valve member 136 and the cage 134, and specifically the plate 190 of the valve member 136 and a surface 194 of the cage 134.

The tube 192 is an exemplary structure or partition that may be included in the valve member 136 to guide the flow of more than one fluid at a time. In particular, the tube 192 has an opening 196 at a first end 198 (see FIG. 1) and at least one opening 200 at a second end 202 (see FIG. 3); as illustrated, a plurality of openings 200 are provided in the second end 202. While the tube 192 is illustrated as coaxial with the conduit 130, this need not be the case according to all embodiments of the present disclosure.

The tube 192 depends from the plate 190. The tube 192 is configured with an interior surface which is preferably generally cylindrical to define a passage 210 through the tube (see FIG. 1). A series of ribs may extend along a portion of the length and outwardly from an outer surface 212 of the tube 192 to close proximity to the cylindrical wall 140 to define annular gaps or passageways between the tube 192 and the conduit 130. In an embodiment, there may be four ribs spaced equally around the outer circumference of the tube 192 that define four annular passageways.

In operation, when the valve member 136 is biased away from the closed position (through interaction between the tube 192 and a structure of the vaporizer, for example), liquid anesthetic is permitted to flow through openings 220 in the cage 134, the second end 146 of the conduit 130, and the passageways defined between the tube 192 and the conduit 130 into an associated vaporizer. At the same time, the first end 198 of the tube 192 is in fluid communication with a portion of the vaporizer through which vapor and possibly some fluid returns to the receptacle 102. This vapor and possible fluid passes through the opening 196 in the first end 198 of the tube 192, through the passage 210 defined through the tube 192, out of the at least one of the openings 200 in the second end 202 of the tube 192, and into the receptacle 102.

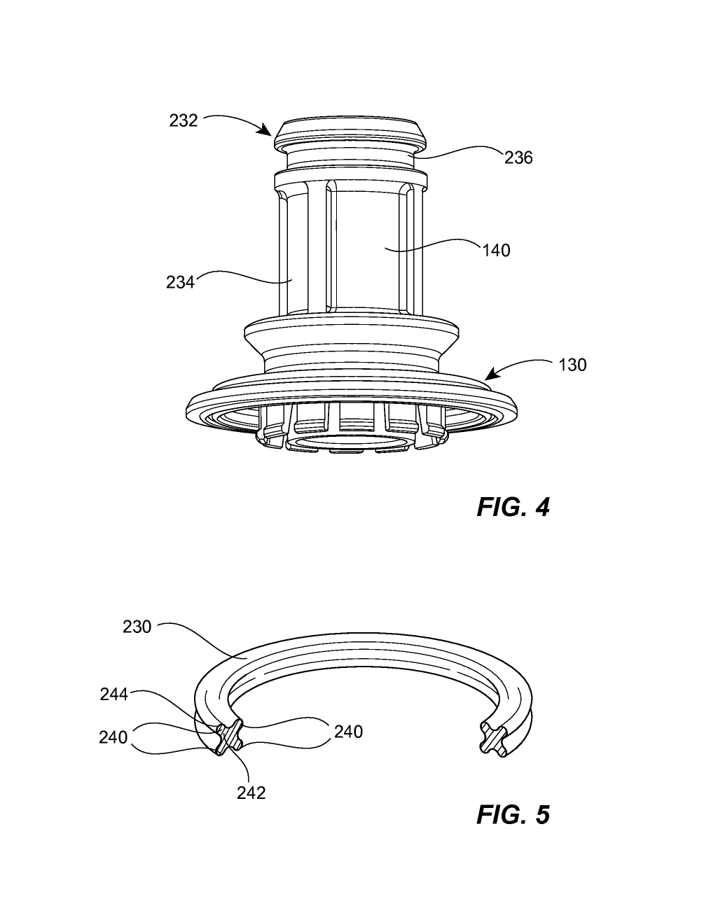

To limit leakage between the valve assembly 104 (and particularly, the conduit 130) and a vaporizer port, a sealing member 230 is disposed about a first end 232 of the conduit 130. In particular with reference to FIGS. 1 and 4, the wall 140 of the conduit 130 has an exterior surface 234 with at least one groove 236 formed therein at the first end 232. The exterior surface 234 may also include other features, such as ribs to improve the rigidity of the conduit 130, for example. As seen in FIG. 1, a toroidal sealing member 230 is disposed in the at least one groove 236, and depends radially outward (relative to the longitudinal axis of the valve assembly 104) from the exterior surface 234 of the conduit 130. In use, the sealing member 230 abuts an inner surface of a passage of a vaporizer port with the conduit 130 disposed through the passage to limit the passage of fluids between the conduit 130 and the vaporizer port.

As best seen in FIGS. 1 and 5, the toroidal sealing member 230 has an X-shaped cross-section, and may be referred to as a quad-ring. The X-shaped cross-section may have four equal lobes 240, with each of the lobes 240 having a first end 242 joined to the first ends 242 of the other lobes 240 and a second, rounded end 244. The sealing member 230 may be made of rubber, and in particular ethylene propylene diene monomer (EPDM) rubber.

The sealing member 230 provides twice the sealing surface in comparison with a conventional o-ring. Further, less force is required to maintain an effective seal with the sealing member 230 relative to the conventional o-ring. The reduction in force required to maintain a fluid-tight seal relative to conventional technology leads to a consequential reduction in friction forces generated when the valve assembly 104 is introduced into and withdrawn from a vaporizer port. The reduction in friction forces leads to a increase in the effective life of the sealing member 230.

It will be appreciated that the advantageous structures of the valve assembly and the toroidal sealing member disclosed above need not both be present in every container according to the present disclosure. That is, the illustrated container includes a valve assembly with a gasketless interface between the valve member and the valve seat, the valve member and the valve seat having sloped surfaces with different slopes. In addition, the conduit of the valve assembly has an exterior sealing member that is in the form of a toroidal sealing member having an X-shaped cross-section. However, in a particular embodiment according to the present disclosure, the gasketless interface between the valve member and the valve seat may be used with a conventional exterior sealing member (e.g., an O-shaped cross-section o-ring). Similarly, the exterior sealing member with an X-shaped cross-section may be used without the gasketless interface defined by the sloped valve member and valve seat having different slopes; other gasketless interfaces may be used, or a gasket may be introduced between the valve member and valve seat, for example. The illustrated embodiment is not intended to limit the claims, but to illustrate the manifold advantages of a container including both gasketless interfaces and the particular sealing member selections according to the present disclosure.

Although the preceding text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

What is claimed is:

1. An anesthetic container comprising:
a receptacle for holding an anesthetic and having a wall defining a interior space and a passage in fluid communication with the interior space; and
a valve assembly that is attachable to the receptacle to control flow of anesthetic through the passage to a vaporizer,
the valve assembly comprising a valve seat with a first sloped surface and a valve member with a second sloped surface, the first sloped surface and the second sloped surface having different slopes,
the first and second sloped surfaces abutting each other to form a gasketless, fluid-tight seal therebetween with the valve assembly in a closed configuration to limit the flow of the anesthetic from the receptacle,
the valve member spaced from the valve seat with the valve assembly in an open configuration to permit the flow of the anesthetic from the receptacle.

2. The anesthetic container according to claim 1, wherein the second sloped surface has a slope that is steeper than a slope of the first sloped surface.

3. The anesthetic container according to claim 1, wherein the valve seat is a frusto-conical surface comprising the first sloped surface; and the valve member comprises a poppet valve with the second sloped surface.

4. The anesthetic container according to claim 3, wherein the poppet valve includes a third sloped surface, the third sloped surface having a slope that is different than the slope of the second sloped surface.

5. The anesthetic container according to claim 1, wherein the valve assembly comprises a conduit having a wall defining a conduit passage with a first end and a second end, the valve seat defined at the second end of the conduit passage.

6. The anesthetic container according to claim 5, wherein the conduit comprises a conduit flange depending outward from the wall, and the valve assembly comprises a cage having a cage flange that is disposed between the conduit flange and the receptacle.

7. The anesthetic container according to claim 6, further comprising a biasing member disposed between the valve member and the cage.

8. The anesthetic container according to claim 5, the wall of the conduit having an external surface with a groove formed therein at the first end, and further comprising a toroidal sealing member disposed in the groove, the toroidal sealing member having an X-shaped cross-section.

9. The anesthetic container according to claim 8, wherein the cross-shaped X-section has four equal lobes.

10. The anesthetic container according to claim 1, further comprising a halogenated inhalation anesthetic contained within receptacle, the anesthetic selected from the group of halogenated anesthetics consisting of sevoflurane, desflurane, isoflurane, enflurane, methoxyflurane and halothane.

11. The anesthetic container according to claim 1, wherein the wall of the receptacle has an exterior surface and comprises a layer of low-density polyethylene that defines at least in part the exterior surface of the wall.

12. An anesthetic container comprising:
a receptacle for holding an anesthetic and having a wall defining a interior space and a passage in fluid communication with the interior space, the anesthetic selected from the group of halogenated anesthetics consisting of sevoflurane, desflurane, isoflurane, enflurane, methoxyflurane and halothane; and
a valve assembly that is attachable to the receptacle to control flow of anesthetic through the passage to a vaporizer,
the valve assembly comprising a conduit having a wall defining a conduit passage with a first end and a second end, a valve seat defined at the second end of the conduit passage and a valve member, the valve member and the valve seat abutting each other in a closed configuration to limit the flow of the anesthetic from the receptacle, and spaced from each other with the valve assembly in an open configuration to permit the flow of the anesthetic from the receptacle,
the wall of the conduit having an external surface with a groove formed therein at the first end, and further comprising a toroidal sealing member disposed in the groove, the toroidal sealing member having an X-shaped cross-section.

13. The anesthetic container according to claim 12, wherein the X-shaped cross-section has four equal lobes.

14. The anesthetic container according to claim 13, wherein each of the four equal lobes has a first end joined to the first ends of the other lobes and a second, rounded end.

15. The anesthetic container according to claim 12, wherein the sealing member comprises rubber.

16. The anesthetic container according to claim 15, wherein the rubber comprises ethylene propylene diene monomer (EPDM) rubber.
